(19) United States
(12) Patent Application Publication
(10) Pub. No.: US 2008/0038840 A1
(43) Pub. Date: Feb. 14, 2008

(54) Fluorescent Method And Compounds To Monitor Protein-Lipid Binding

(75) Inventors: Carsten Schultz, Heidelberg (DE); Oliver Wichmann, Heidelberg (DE); Shannon Black, Auckland (NZ)

Correspondence Address: CONNOLLY BOVE LODGE & HUTZ, LLP, P O BOX 2207, WILMINGTON, DE, 19899, US

(73) Assignee: EUROPAEISCHES LABORATORIUM FUER MOLEKULARBIOLOGIE, HEIDELBERG (DE)

(21) Appl. No.: 11/596,383

(22) PCT Filed: May 13, 2005
(86) PCT No.: PCT/EP05/05274

§ 371 Date: Jan. 12, 2007

(30) Foreign Application Priority Data
May 14, 2004 (EP) 04011596.6

(51) Int. Cl. G01N 33/53; C07D 265/34; G01N 33/92; C07D 279/14

(52) U.S. Cl. 436/501; 436/71; 544/31; 544/99

(57) ABSTRACT

The invention refers to compounds according to formula (I), wherein R¹ is independently in position 2′, 3′, or in both positions of the A ring and is hydroxyl, linear or branched -0-alkyl, wherein the alkyl group has 1-12 carbon atoms, preferably 3-8, more preferably 4 carbon —O-(alkyl)COOH wherein the alkyl group has 1-12 carbon atoms, preferably 3-8, more preferably 4 carbon atoms, —O-(aryl)COOH, -0-aryl, linear or branched —O—(CH₂)ₘ—CH(NH₂)—COOH with m=1-6, preferably m=2-4, or any combination thereof; R⁴ is in position 8′, 10′, or 11′ of the D ring of the compound and is H or (CH₂)ₙ—CH₃ with n=0-6, COOH, CN, or halogen, R⁵ and R⁶ independently are ethyl-, methyl-, H, or —(CH₂)₃ connected to position 8 or 10 of the D ring, and wherein provided that X is O, R²is F, Cl, Br, I, H, CN, —CH═CH-alkyl, —C═—C-alkyl, aryl and R³ is F, Cl, Br, I, CN, —CH═CH-alkyl, —C═C-alkyl, aryl an provided that X is S, R² and R³ independently are F, Cl, Br, I, H, CN, —CH═CH-alkyl, —C═C-alkyl, aryl. The invention further refers to the use of the compounds according to formula (I), wherein R¹, R⁴, R⁵ and R⁶ are defined as aforementioned and wherein R² and R³ independently are F, Cl, Br, I, H, CN, —CH═CH-alkyl, —C═—C-alkyl, or aryl, and wherein X is O or S, as environmentally sensitive fluorescent dyes, particularly in an assay for identifying proteinlipid interactions.

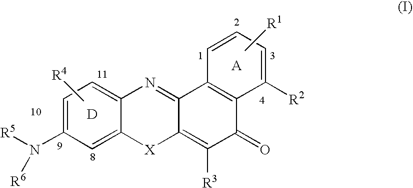

The invention refers to compounds according to formula I

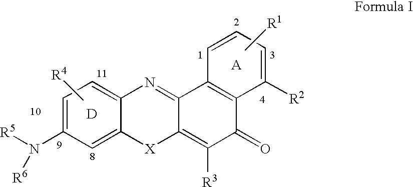

wherein

R¹ is independently in position 2′, 3′, or in both positions of the A ring and is hydroxyl, linear or branched, cyclic or acyclic —O-alkyl, wherein the alkyl group has 1-12 carbon atoms, preferably 3-8, more preferably 4 carbon atoms, —O-(alkyl)COOH wherein the alkyl group has 1-12 carbon atoms, preferably 3-8, more preferably 4 carbon atoms, —O-(aryl)COOH, —O-aryl, linear or branched —O—(CH₂)ₘ—CH(NH₂)—COOH with m=1-6, preferably m=2-4, or any combination thereof;

R⁴ is in position 8′, 10′, or 11′ of the D ring of the compound and is H or (CH₂)ₙ‘3 CH₃ with n=0-6, COOH, CN, or halogen,

R⁵ and R⁶ independently are ethyl-, methyl-, H, or —(CH₂)₃ connected to position 8 or 10 of the D ring,

and wherein

provided that X is O,

R² is F, Cl, Br, I, H, CN, —CH═CH-alkyl, —C≡C-alkyl or aryl and

R³ is F, Cl, Br, I, CN, —CH=CH-alkyl, —C≡C-alkyl or aryl

and

provided that X is S,

R² and R³ independently are F, Cl, Br, I, H, CN, —CH=CH-alkyl, —C≡C-alkyl or aryl.

The invention further refers to the use of the compounds according to formula I as environmentally sensitive fluorescent dyes.

Another subject-matter of the invention is the use of the fluorescent compounds according to the above formula I, wherein

R¹ is independently in position 2′, 3′, or in both positions of the A ring and is hydroxyl, linear or branched, cyclic or acyclic —O-alkyl, wherein the alkyl group has 1-12 carbon atoms, preferably 3-8, more preferably 4 carbon atoms, —O-(alkyl)COOH wherein the alkyl group has 1-12 carbon atoms, preferably 3-8, more preferably 4 carbon atoms, —O-(aryl)COOH, —O-aryl, a linear or branched —O—(CH₂)ₘ—CH(NH₂)—COOH with m=1-6, preferably m=2-4, or any combination thereof;

R² and R³ independently are F, Cl, Br, I, H, CN, —CH=CH-alkyl, —C≡C-alkyl, or aryl,

X is O or S,

R⁴ is in position 8′, 10′, or 11′ of the D ring of the compound and is H or (CH₂)ₙ—CH₃ with n=0-6, COOH, CN, or halogen

R⁵ and R⁶ independently are ethyl-, methyl-, H, or —(CH₂)₃ connected to position 8 or 10 of the D ring,

to identify lipid binding sites on proteins.

The term “aryl” (“O-aryl” in the definition of R¹ and “aryl” in the definition of R² and R³) is defined as mononuclear or binuclear aromatic residue comprising 5- to 7-membered cyclic aromatic groups optionally having one or more heteroatoms from the group N, S or O, which heteroatom may be identical or different, if more than one heteroatom is present, in the ring.

The term “alkyl” in the definition of R² and R³ means an alkyl group with 1-12 carbon atoms, preferably an alkyl group with 4 carbon atoms.

The above-mentioned term “or any combination thereof” preferably means that in case two substituents R¹ are present, both substituents can be the same or can be different from each other.

In one embodiment of the present invention, the use of compounds according to formula I, wherein R¹ is 2′ or 3′-hydroxyl or 2′—O—(CH₂)₅—COOH, R², R³, and R⁴ are H, X is O, and R⁵ and R⁶ are ethyl, to identify lipid binding sites on proteins, are excluded.

The above fluorescent compounds can also be used in an assay for identifying lipid binding sites on proteins and for measuring the binding of ligands to lipid binding sites of proteins.

Binding of proteins to lipids is a crucial event in translocation of proteins within the cell, e.g. from the cytosol to the mitochondrial matrix, as well as lipid and drug transport. The proteins usually have hydrophobic cavities referred to as lipid binding sites. From the amino acid sequence the specificity of the binding site for a particular lipid cannot be deduced. Especially in the post-genomic era it is important to determine protein-lipid interaction in order to find physiological functions of proteins. Due to the heterogeneous nature of biological membranes, the specificity of lipid-protein interactions is difficult to investigate with native material. Typically, artificial membranes or their substitutes like liposomes or micelles are employed instead, which may lead to unsatisfactory results. It would be desirable to study protein-lipid interaction by adding a single species to the protein.

The study of specific lipid-protein binding is particularly crucial under pharmacological aspects, since many drugs bind to physiological proteins such as albumin, thus replacing endogenous ligands as well as other drugs which can lead to strong, unpredictable side effects and in addition affects the pharmacokinetic properties of the drug.

One approach to investigate specific lipid-protein interaction involves environmentally sensitive fluorescent dyes that change fluorescence properties depending on the hydrophobicity of the surroundings. A known fluorescent dye with these properties is nile red, a dye usually employed to stain lipid droplets in cells. The structure of Nile red forms the basis of formula I with R¹, R², R³, and R⁴═H, and R⁵ and R⁶=ethyl.

A main disadvantage of nile red in studying lipid-protein interaction in whole cells is a strong quenching effect of the fluorescence by cytochrome c (Bertsch et al., 2003, Analytical Biochemistry 313:187-195). In addition, measurement of lipid-protein interaction with nile red in vitro is hindered by the fact, that nile red is not water-soluble and the fluorescence spectrum is not convenient for automated measurement.

The object of the invention is thus to provide environmentally sensitive fluorescent dyes which alter the emission wavelength depending on the hydrophobicity of the surroundings and which can be used for the determination of lipid-protein interactions in an easy, cheap and very fast high-throughput-screening (HTS) assay in vivo or in vitro.

This object is solved by providing compounds according to formula I

wherein

R¹ is independently in position 2′, 3′, or in both positions of the A ring and is hydroxyl, linear or branched, cyclic or acyclic —O-alkyl, wherein the alkyl group has 1-12 carbon atoms, preferably 3-8, more preferably 4 carbon atoms, —O-(alkyl)COOH wherein the alkyl group has 1-12 carbon atoms, preferably 3-8, more preferably 4 carbon atoms, —O-(aryl)COOH, —O-aryl, linear or branched —O—(CH₂)ₘ—CH(NH₂)—COOH with m=1-6, preferably m=2-4, or any combination thereof;

R⁴ is in position 8′, 10′, or 11′ of the D ring of the compound and is H or (CH₂)ₙ—CH₃ with n=0-6, COOH, CN, or halogen,

R⁵ and R⁶ independently are ethyl-, methyl-, H, or —(CH₂)₃ connected to position 8 or 10 of the D ring,

and wherein

provided that X is O,

R² is F, Cl, Br, I, H, CN, —CH═CH-alkyl, —C≡C-alkyl, or aryl and

R³ is F, Cl, Br, I, CN, —CH═CH-alkyl, —C≡C-alkyl, or aryl

and

provided that X is S,

R² and R³ independently are F, Cl, Br, I, H, CN, —CH═CH-alkyl, —C≡C-alkyl, or aryl.

The above fluorescent compounds are able to change their fluorescence properties depending on the hydrophobicity of the environment, characterised in that they show excitation and emission at wavelengths that are longer than those of e.g. unsubstituted nile red, facilitating their use in automatic applications.

Furthermore, the fluorescent dyes according to the invention containing hydrophilic substituents are water-soluble at the concentrations employed in the competitive binding assay to measure binding of ligands to lipid binding sites of proteins according to the invention. In addition, all fluorescent dyes according to the invention with R⁵ and R⁶ both H can be bound covalently to glass or plastic surfaces of e.g. multi well plates, plate reader plates, micro array chips or microfluidic devices. These features both contribute to the convenient use of the fluorescent dyes according to formula I in automatic applications.

The object of the invention is further solved by providing an assay for identifying lipid binding sites on proteins using a fluorescent compound according to formula I, wherein R¹, R⁴, R⁵ and R⁶ are defined as above-mentioned and wherein X is O or S and wherein R² and R³ independently are F, Cl, Br, I, H, CN, —CH═CH-alkyl, —C≡C-alkyl, or aryl. The invention further provides a competitive binding assay making use of the above-mentioned fluorescent compounds to measure ligand binding to lipid binding sites of proteins. Both assays can be performed either with the fluorescent dyes in aqueous solution or with the fluorescent dyes covalently bound to the surfaces mentioned above.

The object is further solved by providing a competitive binding assay according to the invention using the new environmentally sensitive fluorescent dyes which is suitable for an easy, cheap, and very fast high throughput screening for ligands binding to lipid binding sites of proteins.

Increased water-solubility of the fluorescent dyes according to formula I as compared to e.g. nile red, was achieved by coupling them via 2′-O- or 3′-O- on the A-ring of the fluorescent dye with fatty acids, or amino acids of variable lengths (Briggs et al. (1997) J. Chem. Soc., Perkin Trans., 1:1051). Butyric acid derivatives are preferably used in an assay to identify lipid binding sites on proteins, due to its sufficient water solubility. An example for such a butyric acid derivative showing an increased water solubility is nile red butyric acid (NRBA), wherein in formula I R¹ is in position 2′ of the A ring of the fluorescent dye and is —O—(CH₂)₃—COOH, R², R³, and R⁴ each are H, X is O, and R⁵ and R⁶ each are ethyl.

The insertion of halogen atoms for instance at the R³-position on the fluorescent dye leads to a shift of the excitation/emission of that dye towards longer wavelengths. The use of fluorescent dyes with longer wavelengths compared to those with shorter wavelengths has the advantage that thereby the interference of the fluorescent signal of the dye by the autofluorescence of the tested protein (in particular by the tryptophane residues of the protein) or by the autofluorescence of the other ligands or cofactors of the protein (such as flavonoids or heme complexes) will be limited. In so far, the use of fluorescent dyes with inserted halogen atoms in the assay of the invention will help to minimize the potential overlap between the excitation and/or emission spectra of the tested protein (including its cofactors) and the excitation and/or emission spectra of the fluorescent dye itself. Analogously, substitution of the benzo[a]phenoxazin by benzo[a]phenothiazin and/or the further insertion of a methyl group or any other electron donor group on the D-ring of the fluorescent dye leads to a comparable shift of the excitation/emission of that dye to longer wavelengths.

Fluorescent dyes with longer excitation and/or emission wavelengths which show a minimal overlap with the excitation and/or emission wavelength of the tested protein or of the tested protein complex are particularly preferred for the performance of an automated large-scale assay, in particular for an automated assay, wherein the extent of the measured protein-lipid-binding of different tested proteins will be quantified and compared to each other.

One preferred embodiment of the invention exemplifying halogenation includes thus compounds according to formula I, wherein R¹ is independently in position 2′, 3′ or both of the A ring of the fluorescent dye and is hydroxyl, linear or branched —O-alkyl, wherein alkyl has 1-12 carbon atoms, preferably 3-8, more preferably 4 carbon atoms, —O-carboxylic acid with 1-12 carbon atoms, preferably 3-8, more preferably 4 carbon atoms, —O-aryl, linear or branched —O—(CH₂)ₘ—CH(NH₂)—COOH with m=1-6, preferably m=2-4, or any combination thereof,

wherein R² and R³ are independently F, Cl, Br, I or H, with the proviso that R² and R³ are not simultaneously hydrogen, wherein X is O, R⁴ is H, and R⁵ and R⁶ independently are ethyl-, methyl-, H, or —(CH₂)₃ connected to position 8 or 10 of the D ring of the fluorescent dye.

Particularly preferred are compounds according to formula I, wherein R¹, X, R⁴, R⁵ and R⁶ are defined as above and wherein R² and R³ independently are F, Cl, Br, I, H, CN, —CH═CH-alkyl, —C≡C-alkyl, or aryl.

Examples for fluorescent dyes containing halogen atoms are neckar yellow butyric acid (NYBA), wherein in formula I R¹ is in position 2′ of the A ring of the fluorescent dye and is —O—(CH₂)₃—CH₃, R² and R⁴ each are H, R³ is F, X is O, and R⁵ and R⁶ each are ethyl; fluorescent dyes according to formula I, wherein R¹ is in position 3′ of the A ring of the fluorescent dye and is hydroxyl, linear or branched —O-alkyl with 1-12 carbon atoms, preferably 3-8, more preferably 4 carbon atoms, —O-carboxylic acid with 1-12, preferably 3-8, more preferably 4 carbon atoms, —O-aryl, linear or branched —O—(CH₂)ₘ—CH(NH₂)—COOH with m=1-6, preferably m=2-4, R² and R⁴ each are H, R³ is Br, X is O, and R⁵ and R⁶ each are ethyl (e.g. compound 4 and 9 according to table 1); and fluorescent dyes according to formula I, wherein R¹ is in position 3′ of the A ring of the fluorescent dye and is hydroxyl, linear or branched —O-alkyl with 1-12 carbon atoms, preferably 3-8, more preferably 4 carbon atoms, —O-carboxylic acid with 1-12 carbon atoms, preferably 3-8, more preferably 4 carbon atoms, an —O-aryl, linear or branched —O—(CH₂)ₘ—CH(NH₂)—COOH with m=1-6, preferably m=2-4, R² and R³ each are Br, R⁴ is H, X is O, and R⁵ and R⁶ each are ethyl (e.g. compound 6 according to table 1).

Another preferred embodiment of the invention exemplifying the substitution of the benzo[a]phenoxazin by benzo[a]phenothiazin and the further insertion of any electron donor group on the D-ring of the fluorescent dye includes compounds according to formula I, wherein R¹ is in position 2′, 3′ or both of the A ring of the fluorescent dye and is hydroxyl, linear or branched —O-alkyl, wherein alkyl has 1-12 carbon atoms, preferably 3-8, more preferably 4 carbon atoms, —O-carboxylic acid with 1-12 carbon atoms, preferably 3-8, more preferably 4 carbon atoms, an —O-aryl, linear or branched —O—(CH₂)ₘ—CH(NH₂)—COOH with m=1-6, preferably m=2-4, or any combination thereof, wherein X is S, R² and R³ independently are F, Cl, Br, I, H, CN, —CH═CH-alkyl, —C≡C-alkyl, or an aryl, R⁴ is H or (CH₂)ₙ—CH₃ with n=0-6, COOH, CN, or halogen, and R⁵ and R⁶ independently are ethyl-, methyl-, H, or —(CH₂)₃ connected to position 8 or 10 of the D ring of the fluorescent dye. Examples for this preferred embodiment are fluorescent dyes according to formula I, wherein R¹ is in position 3′ of the A ring of the fluorescent dye and is hydroxyl, linear or branched —O-alkyl with 1-12 carbon atoms, preferably 3-8, more preferably 4 carbon atoms, —O-carboxylic acid with 1-12 carbon atoms, preferably 3-8, more preferably 4 carbon atoms, an —O-aryl, linear or branched —O—(CH₂)ₘ—CH(NH₂)—COOH with m=1-6, preferably m=2-4, R² and R³ each are H, R⁴ is methyl, X is S, and R⁵ and R⁶ each are ethyl (e.g. compounds 6, 7 and 9 according to table 1).

A further preferred embodiment comprises an assay for identifying lipid binding sites of proteins making use of the environmentally sensitive fluorescent dyes according to formula I, wherein R¹ is independently in position 2′, 3′, or both of the A ring of the fluorescent dye and is hydroxyl, linear or branched —O-alkyl with 1-12 carbon atoms, preferably 3-8, more preferably 4 carbon atoms, —O-carboxylic acid with 1-12 carbon atoms, preferably 3-8, more preferably 4 carbon atoms, an —O-aryl, linear or branched —O—(CH₂)ₘ—CH(NH₂)—COOH with m=1-6, preferably m=2-4, or any combination thereof, R² and R³ independently are F, Cl, Br, I, H, CN, —CH═CH-alkyl, —C≡C-alkyl, aryl, X is O or S, R⁴ is H or (CH₂)ₙ—CH₃ with n=0-6, COOH, CN, or halogen, and R⁵ and R⁶ each are H. Another embodiment comprises compounds, wherein R¹ and R⁴ are defined as above and wherein, provided that X is O, R¹ is F, Cl, Br, I, H, CN, —CH═CH-alkyl, —C≡C-alkyl, aryl and R³ is F, Cl, Br, I, CN, —CH═CH-alkyl, —C≡C-alkyl, aryl and, provided that X is S, R² and R³ independently are F, Cl, Br, I, H, CN, —CH═CH-alkyl, —C≡C-alkyl, aryl and wherein R⁵ and R⁶ each are H. These aforementioned fluorescent dyes, wherein R⁵ and R⁶ each are H, can be covalently bound via a spacer coupled to the amino group of the D-ring of the fluorescent dye to glass or plastic surfaces of for example multi well plates, plate reader plates, micro array chips, or microfluidic devices, which can then be employed for high throughput screening for ligands binding to lipid binding sites of proteins.

The environmentally sensitive fluorescent dyes according to the present invention are synthesized using standard methods as described exemplary in examples 1 to 5. Compounds with a 6-Bromo- or 6-Chloro-substitution could be used to make compounds with a 6-Fluoro or 6-Iodo substitution as well. These halogenated fluorescent dyes particularly the iodo-, chloro- and bromo-compounds) could be used for the synthesis of further fluorescent dyes via Suzuki, Stille or Sonogashira methodology.

Some exemplary fluorescent dyes according to the invention together with values for excitation and emission are included in table 1. For compound 1, which is the 2′-hydroxy derivative of nile red (with X═O), the excitation and emission wavelengths are significantly shorter than for the other listed compounds of the invention with halogen ligands and/or with X═S (No. 2-10). The excitation and emission values of the fluorescent dyes were measured in methanol, which simulates a protein-environment, thus yielding a shift of the emission to shorter wavelengths. For comparison, excitation and emission values for compound 10 were also measured in HEPES buffer (i.e. under initial, aqueous assay conditions). In this aqueous environment, the excitation is 620 nm and the emission is 688 nm compared to an emission at 662 nm in methanol.

TABLE 1
Examples of fluorescent dyes according to the invention together with molecular weights and wavelengths for excitation and
emission

| | | Excitation | Emission |
|---|---|---|---|
| Structure | Molecular Weight (U) | (nm) | (nm) |
| 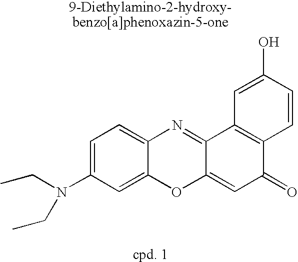 | C₂₀H₁₈N₂O₃Exact Mass: 334.13 | 540 | 629 |
| 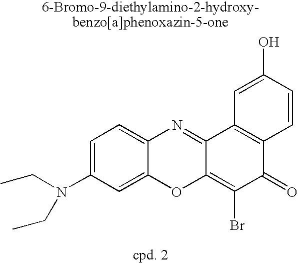 | C₂₀H₁₇BrN₂O₃Exact Mass: 412.04 | 550 | 640 |
| 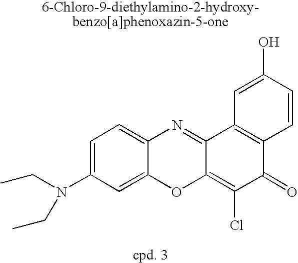 | C₂₀H₁₇CIN₂O₃Exact Mass: 368.09 | 549 | 643 |
| 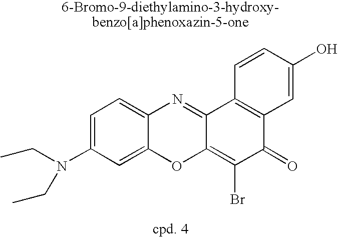 | C₂₀H₁₇BrN₂O₃Exact Mass: 412.04 | 552 | 650 |
| 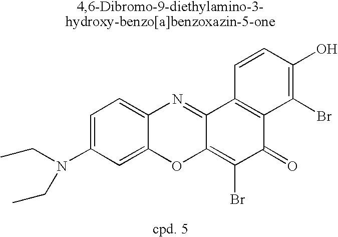 | C₂₀H₁₆Br₂N₂O₃Exact Mass: 489.95 | 562 | 653 |
| 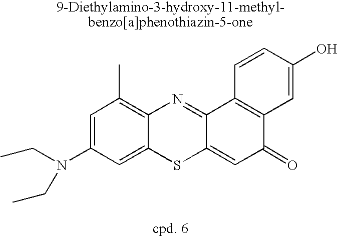 | C₂₁H₂₀N₂O₂S Exact Mass: 364.12 | 580 | 665 |
| 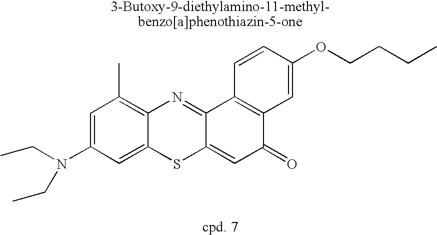 | C₂₅H₂₈N₂O₂S Exact Mass: 420.19 | 585 Quantum Yield 0.29 | 664 |
| 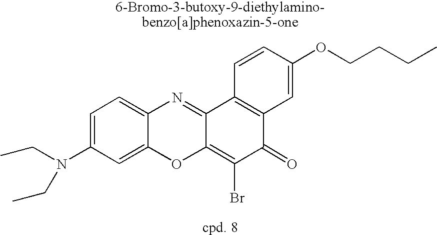 | C₂₄H₂₅BrN₂O₃Exact Mass: 468.10 | 556 Quantum Yield 0.35 | 650 |
| 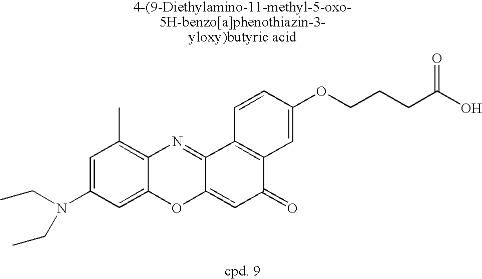 | C₂₅H₂₆N₂O₄S Exact Mass: 450.16 | 580 Quantum Yield 0.30 | 662 |
| 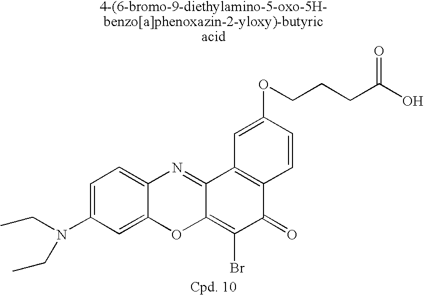 | C₂₄H₂₅BrN₂O₅Exact Mass: 498.08 | 549 | 642 |
| 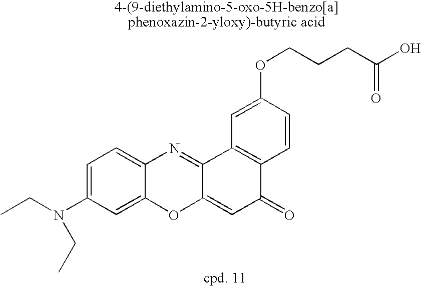 | C₂₄H₂₄N₂O₅Exact Mass: 420.17 | 544 | 631 |

The invention further relates to an assay for identifying lipid binding sites of proteins using the compounds according to formula I, wherein

R¹ is independently in position 2′, 3′, or in both positions of the A ring and is hydroxyl, linear or branched —O-alkyl, wherein the alkyl group has 1-12 carbon atoms, preferably 3-8, more preferably 4 carbon atoms, —O-(alkyl)COOH, wherein the alkyl group has 1-12 carbon atoms, preferably 3-8, more preferably 4 carbon atoms, —O-(aryl)COOH, —O-aryl, a linear or branched —O—(CH₂)ₘ—CH(NH₂)—COOH with m=1-6, preferably m=2-4, or any combination thereof;

R² and R³ independently are F, Cl, Br, I, H, CN, —CH═CH-alkyl, —C≡C-alkyl, or aryl,

X is or S,

R⁴ is in position 8′, 10′, or 11′ of the D ring of the compound and is H or (CH₂)ₙ—CH₃ with n=0-6, COOH, CN, or halogen

R⁵ and R⁶ independently are ethyl-, methyl-, H, or —(CH₂)₃ connected to position 8 or 10 of the D ring.

In a preferred embodiment of the invention, the assay for identifying lipid binding sites of proteins using the compounds according to formula I as defined above, comprises the steps of
a) dissolving a compound according to formula I as defined above in an aqueous solvent,
b) adding the protein to be tested for containing a lipid binding site, preferably in various concentrations,
c) measuring the intensity of fluorescence emission at the wavelength of maximal emission of the fluorescent dye.

The fluorescent dye will bind to any lipid-binding site of the protein, and the resulting increase in the hydrophobicity surrounding of the dye will result in an increase in the intensity of the fluorescence and a shift in the emission to a shorter wavelength. The emission-wavelength of a compound according to formula I in a hydrophobic surrounding can be determined in a non-aqueous solution such as methanol.

The invention further relates to a competitive binding assay to measure the binding of ligands to lipid binding sites of proteins using the compounds according to formula I as defined above comprising the steps of
a) dissolving a compound according to formula I as defined above in an aqueous solvent,
b) adding a protein containing at least one lipid binding site,
c) adding the ligand to be tested, preferably in various concentrations,
d) measuring the intensity of fluorescence emission at the wavelength of maximal emission of the fluorescent dye.

Depending on the strength of the binding of the ligand to the lipid binding site of the protein, the ligand releases the fluorescent dye from the hydrophobic lipid binding site. This can be measured by a decrease of the intensity of fluorescence at the emission wavelength of the dye in hydrophobic surrounding, as imitated by dissolving the dye in methanol.

In a preferred embodiment of the competitive binding assay, a fluorescent dye covalently linked to glass or plastic surfaces of for example multi-well-plates, plate-reader-plates, micro array chips or microfluidic devices is applied in step a) of the assay, in step b) the surface is loaded with a protein containing at least one lipid binding site, step c) is carried out as described, and in step d) the intensity of fluorescence emission at the wavelength of maximal emission of the fluorescent dye is measured in an appropriate reader.

The present invention further relates to the use of the competitive binding assay for a high throughput screening for ligands which are useful as competitive inhibitors of protein-lipid interactions. These competitive inhibitors can be used wherever a specific lipid-protein interaction leads to a pathophysiological state in patients.

The invention further relates to the use of the competitive binding assay to study pharmacokinetics of drug binding to lipid binding sites on proteins. As mentioned above, many drugs bind to physiological proteins such as albumin, thus releasing endogenous ligands or other drugs which can lead to strong, unpredictable side effects and in addition affects the pharmacokinetic properties of the drug.

Drugs which strongly bind to the lipid binding site of proteins will thus release drugs which show only a weak binding to endogenous proteins, raising the blood levels of the latter. The number of patients, especially the elderly, taking several drugs at a time, often prescribed by different doctors, is increasing. For this reason there is a strong need to study possible interactions of drugs. By comparing the respective binding properties of different drugs using the fluorescent dyes and the assays according to the invention (FIG. 4 and 5), it is possible to predict drug interaction due to competitive binding to the lipid binding sites of endogenous proteins and thus prevent side effects which are prone to occur especially in patients taking several drugs at a time.

Another object of the invention is a kit for identifying competitive inhibitors of a protein lipid interaction comprising a fluorescent compound as defined above, optionally a protein with at least one lipid binding site and optionally a solvent to solubilize the fluorescent dye and the protein.

Another object of the invention is a method of providing a medicament comprising a competitive inhibitor of a protein lipid interaction comprising the steps of
dissolving the fluorescent dye of the invention in an aqueous solvent,
adding a protein containing at least one lipid binding site,
adding the compound to be tested, preferably in various concentrations,
measuring the intensity of fluorescence emission at the wavelength of maximal emission of the fluorescent dye, preferably in non-aquous solution
identifying a compound which is a competitive inhibitor of the protein lipid interaction by detecting an alteration of the emission wavelength or fluorescence intensity of the fluorescent dye
providing the same compound by chemical or biological methods
and manufacturing the compound into a medicament.

“Environmentally sensitive fluorescent dyes” within the meaning of the invention are fluorescent dyes that change fluorescence properties depending on the hydrophobicity of the surroundings.

“Multi well plates” within the meaning of the invention are plates of different material, mostly plastic or glass, comprising at least 6 wells.

“Plate reader plates” within the meaning of the invention are plates of different material, mostly plastic or glass, comprising at least 96 wells, but also several hundred or up to several thousand wells. An example for plate reader plates are micro titre plates.

“Micro array chips” within the meaning of the invention are solid phase carriers of different materials in the form of small-sized chips, e.g. in the size of cover slips.

“Microfluidic devices” within the meaning of the invention are miniaturized modules for unit operations in chemical technology which can be applied in high throughput screening assays.

“High throughput screening” within the meaning of the invention comprises screening assays allowing the screening of at least 96 different compounds or different concentrations of one or several compounds at the same time.

### FIGURES

FIG. 1a shows the titration of 2-hydroxy-6-bromo-nile red butyric acid (2BrNR, cmp.10, [0.25 μM]) carrying halogen ligands with bovine serum albumin (BSA) which leads to a shift in the emission to a shorter wavelength and an increase in the intensity of the fluorescence.

FIG. 1b shows the titration of nile red butyric acid (NRBA, cmp. 11 [3 μM]) carrying no halogen ligands with bovine serum albumin (BSA) which also leads to a shift in the emission to a shorter wavelength and an increase in the intensity of the fluorescence.

Furthermore, it is shown by FIG. 1a and 1b that the maximal emission intensity is observed at wavelength 670 nm for BrNR and at wavelength 657 mn for NRBA in the absence of BSA and at wavelength 642 mn for BrNR and at wavelength 620 nm for NRBA in the presence of BSA. The clear shift to longer emission wavelengths for the Br-substituted BrNR compared to the halogen-free NRBA can therefore be easily concluded from FIG. 1a and 1b.

FIG. 2a shows the titration of 2BrNR, (cmp. 10, [250 nM]) with different proteins: BSA, the peroxisomal matrix protein and monoamine oxidase B (MAO B).

FIG. 2b shows the titration of NRBA [250 nM] with different proteins: BSA, the peroxisomal matrix protein (SCP2), and monoamine oxidase B (MAO B). BSA had the strongest binding to NRBA, SCP2 the weakest.

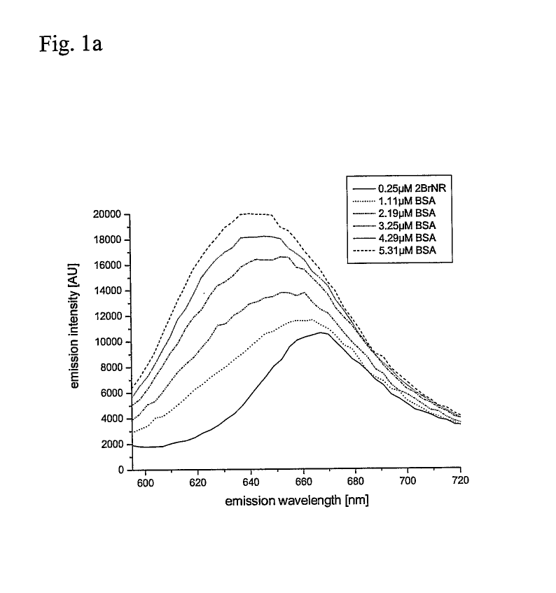

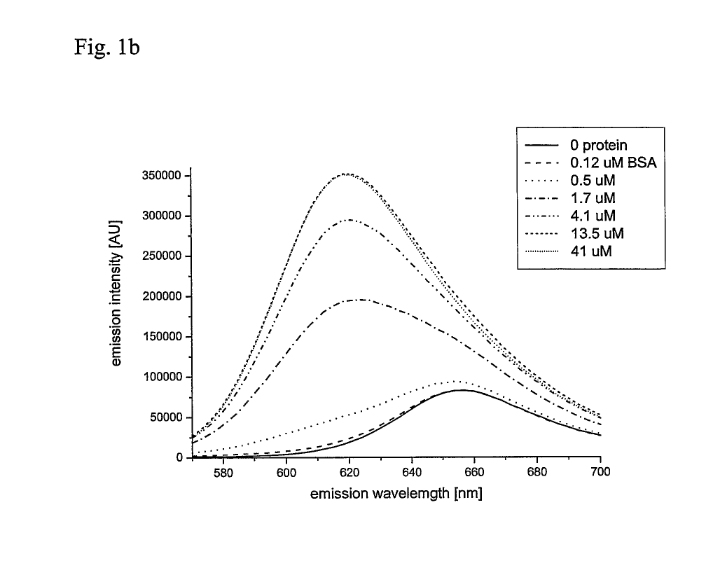

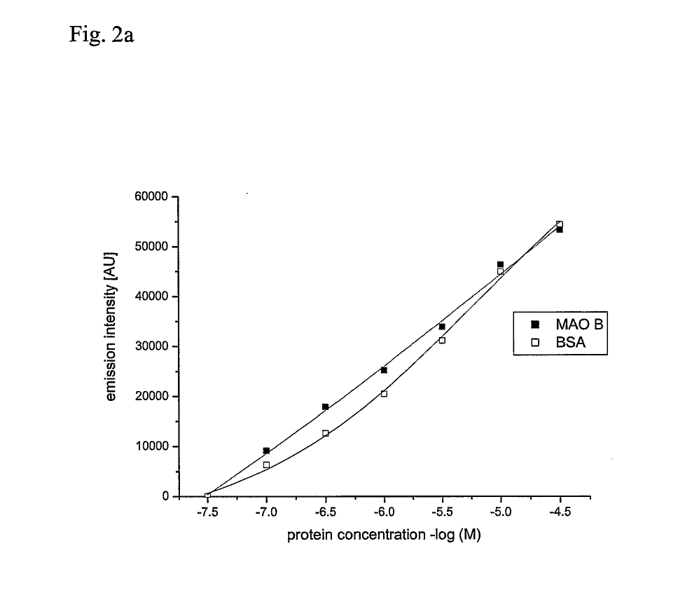

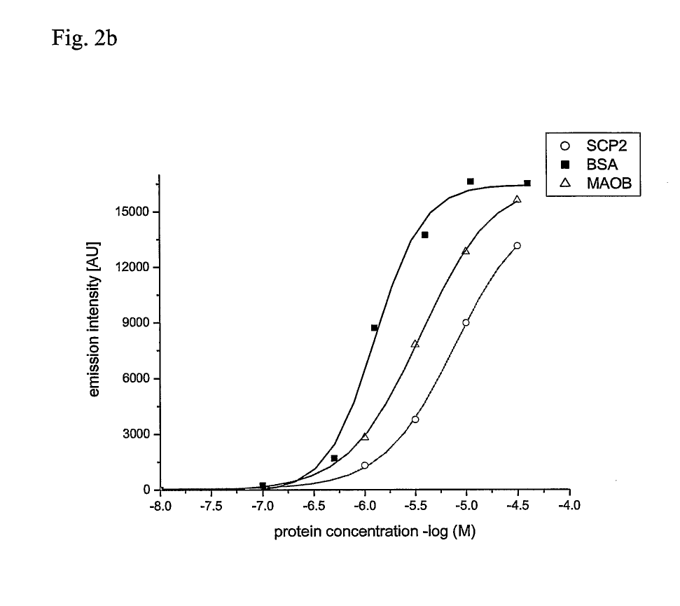

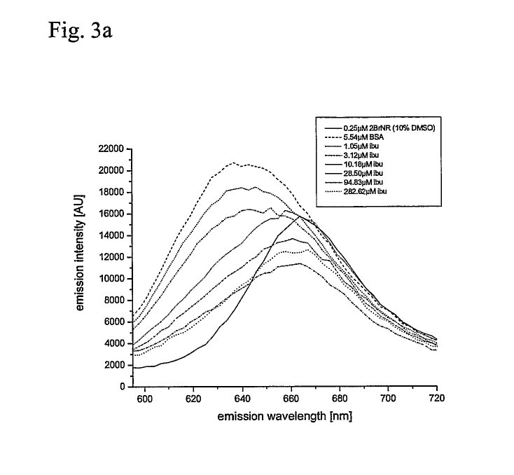

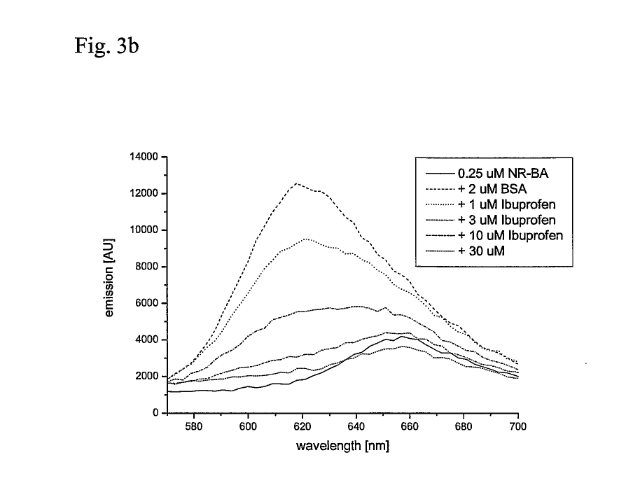

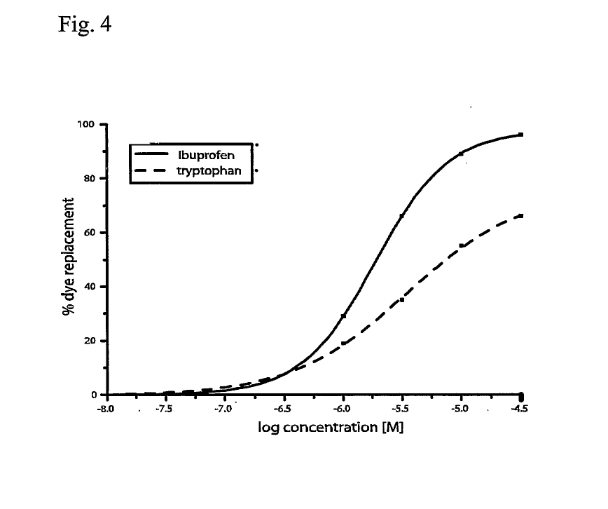

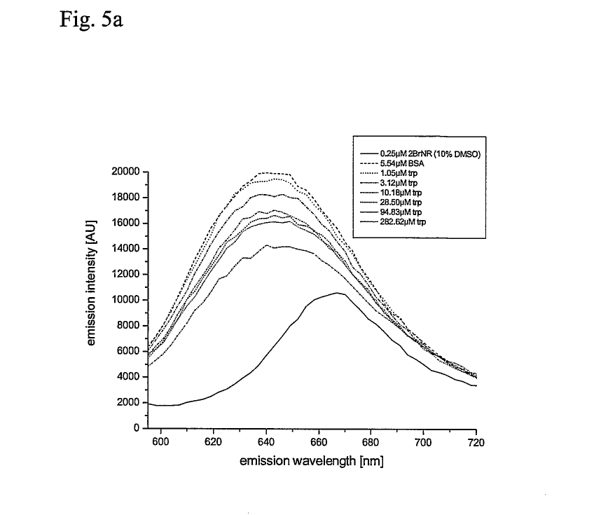

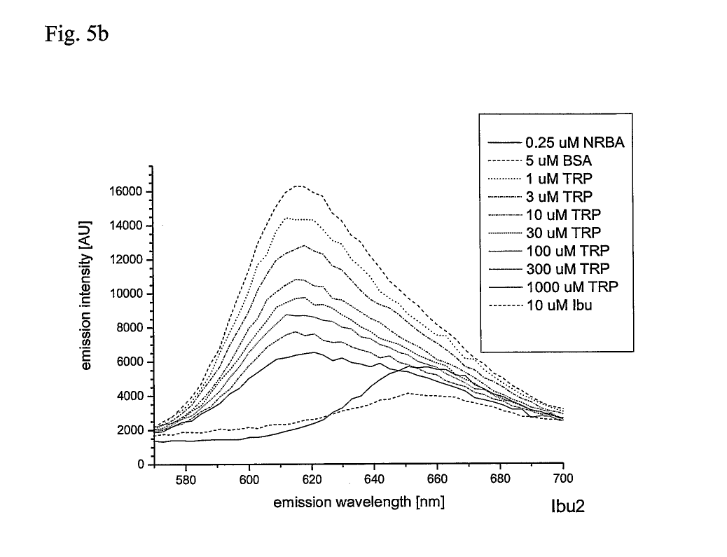

When FIG. 2a and 2b are compared, it becomes apparent that the sensitivity of a competitive assay making use of the bromo-substituted dye BrNR (see FIG. 2a) is large over a broader range of different protein concentrations compared to a competitive assay that uses the halogen-free fluorescent dye NRBA (see FIG. 2b). For instance, the addition of BSA from the negative logarithms of −7,5 to −7,0 or the addition of BSA from the negative logarithms of −5,0 to −4,5 will not result in measurable changes of the emission intensities when NRBA is used (see FIG. 2b), on the other hand, the addition of the same amounts of BSA to a BrNR-solution will lead to a considerable (and measurable) change in the emission intensity of BrNR (see FIG. 2a).

Another advantage of the bromo-substituted fluorescent dye BrNR is that the changes in the emission intensities of BrNR also seem to be larger over a defined range of protein concentrations compared to those of the halogen-free fluorescent dye NRBA. For instance, the emission intensities of BrNR range between 0 and 60000 AU and the emission intensities of NRBA range from 0 to 15000 for the negative logarithm of the protein concentrations of −7,5 to −4,5.

FIG. 3a shows the replacement of 2BrNR [250 nM] from 5.54 μM BSA with ibuprofen.

FIG. 3b shows the replacement of NRBA [250 nM] from 2 μM BSA with ibuprofen.

FIG. 4 shows a comparison of the replacement of NRBA from 2 μM BSA with ibuprofen and tryptophan (the endogenous ligand of albumin). Ibuprofen was able to bind to both lipid binding sites of albumin while tryptophan only replaced one molecule of the dye.

FIG. 5a shows the replacement of 2BrNR [250 nm] from 5.54 μM BSA with tryptophan. In contrast to ibuprofen (see FIG. 3a), even 282.62 μM of tryptophan was not able to release all the dye from BSA.

FIG. 5b shows the experiment of FIG. 4 in a diagram of emission intensity vs. emission wavelength. In contrast to ibuprofen [10 μM], even 1 mM of tryptophan was not able to release all the dye from BSA (BSA titrated with tryptophan showed occupation of one binding site).

### EXAMPLES

### Example 1

### Synthesis of 6-Bromo-9-diethylamino-3-hydroxy-benzo[a]phenoxazin-5-one (compound 4 in table 1)

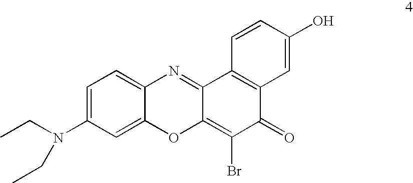

Bromine (0.10 ml, 1.9 mmol) was added to a solution of 9-Diethylamino-3-hydroxy-benzo[a]phenoxazin-5-one (0.20 g, 0.6 mmol) in 100 ml chloroform and the solution allowed to stir at RT, under nitrogen for 2 h. The solution was then concentrated under reduced pressure (30° C.) and the residue further purified by column chromatography [methanol/dichloromethane (3:97)—methanol/dichloromethane (10:90)] to give 6-Bromo-9-diethylamino-3-hydroxy-benzo[a]phenoxazin-5-one (0.06 g, 25%), (FAB⁺) 413 (60%).

### Example 2

### Synthesis of 4,6-Dibromo-9-diethylamino-3-hydroxy-benzo[a]phenoxazin-5-one (Compound 5 in table 1)

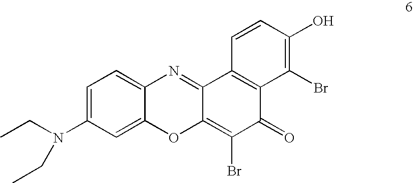

Bromine (0.20 ml, 3.6 mmol) was added to a solution of 9-Diethylamino-3-hydroxy-benzo[a]phenoxazin-5-one (0.20 g, 0.6 mmol) in 100 ml chloroform and the solution allowed to stir at 40° C., under nitrogen for 2 h. The solution was then concentrated under reduced pressure (40° C.) and the residue further purified by column chromatography [methanol/dichloromethane (1:99)—methanol/dichloromethane (5:95)] to give 4,6-Dibromo-9-diethylamino-3-hydroxy-benzo[a]phenoxazin-5-one (0.085 g, 29%), (FAB⁺) 493 (100%).

### Example 3

### Synthesis of 9-Diethylamino-3-hydroxy-11-methyl-benzo[a]phenothiazin-5-one (compound 6 in table 1)

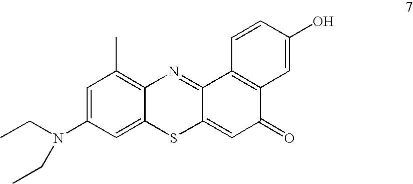

Thiosulfuric acid S-(2-amino-5-diethylamino-3-methyl-phenyl)ester (0.50 g, 1.7 mmol) (Photochem. and Photobiol., 1998, 67, 343) and 1,7 dihydroxynaphthalene (0.28 g, 1.7 mmol) were dissolved in dimethylsulfoxide (10 ml) and stirred at RT for 45 min. Potassium dichromate (0.51 g, 1.73 mmol) was added and the reaction stirred for a further 20 min. A mixture of methanol (200 ml) and 1N HCl (20 ml) was then added and the solution stirred for a further 1 h at RT. The excess methanol was then removed under reduced pressure and aq. sodium bicarbonate was added to the residue in dimethylsulfoxide. The mixture was sealed and left at RT for 18 h. The resultant precipitate was removed by filtration, washed with water and dried under high vacuum to yield 9-Diethylamino-3-hydroxy-11-methyl-benzo[a]phenothiazin-5-one (0.46 g, 73 %), (FAB⁺) 365 (20%).

### Example 4

### Synthesis of 4-(6-Bromo-9-diethylamino-5-oxo-5H-benzo[a]phenoxazin-2-yloxy)-butyric acid (compound 10 in table 1)

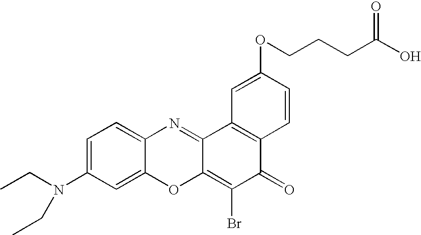

### 4-(9-Diethylamino-5-oxo-5H-benzo[a]phenoxazin-2-yloxy)-butyric acid allyl ester

9-Diethylamino-2-hydroxy-benzo[a]phenoxazin-5-one (1.50 g, 4.5 mmol), 4-bromo-butyric acid allyl ester (1.40 g, 6.8 mmol) and potassium carbonate (3.60 g, 26.0 mmol) were added in DMF (75 ml). The solution was heated at gentle reflux for 7 h. Brine was then added and the solution extracted with ethyl acetate. The combined organic layers were washed with brine, dried and concentrated under reduced pressure. The residue was further purified by column chromatography (dichloromethane) to give 4-(9-diethylamino-5-oxo-5H-benzo[a]phenoxazin-2-yloxy)-butyric acid allyl ester (0.89 g, 43%), (FAB⁺) 461 (80%).

### 4-(6-Bromo-9-diethylamino-5-oxo-5H-benzo[a]phenoxazin-2-yloxy)-butyric acid allyl ester

4-(9-Diethylamino-5-oxo-5H-benzo[a]phenoxazin-2-yloxy)-butyric acid allyl ester (0.08 g, 0.17 mmol) was dissolved in dimethylsulfoxide (5 ml). N-ethyldiisopropylamine (0.05 g, 0.4 mmol) was added, followed by bromomethylacetate (0.05 g, 0.4 mmol). The solution was then stirred at RT for 1.5 h; After the addition of brine, the solution was extracted three times with ethyl acetate. The combined organic fractions were dried and concentrated under reduced pressure. The residue was further purified by column chromatography [dichloromethane—methanol/dichloromethane (2:98)] to give 4-(6-bromo-9-diethylamino-5-oxo-5H-benzo[a]phenoxazin-2-yloxy)-butyric acid allyl ester (0.07 g, 78%), (FAB⁺) 541 (100%), 539 (100%).

### 4-(6-Bromo-9-diethylamino-5-oxo-5H-benzo[a]phenoxazin-2-vloxY)-butyric acid

4-(6-bromo-9-diethylamino-5-oxo-5H-benzo[a]phenoxazin-2-yloxy)-butyric acid allyl ester (0.07 g, 0.14 mmol), diethylamine (0.04 g, 0.5 mmol) and tetrakis(triphenylphosphine)palladium (0.05 g, 0.04 mmol) were added together in dry dimethylformamide (10 ml). The solution was stirred at RT for 18 h. The solvent was removed under reduced pressure and the residue was further purified by column chromatography [dichloromethane—methanol/dichloromethane/acetic acid (6:94:0.01)] to give 4-(6-bromo-9-diethylamino-5-oxo-5H-benzo[a]phenoxazin-2-yloxy)-butyric acid (0.04 g, 51%), (FAB⁺) 501 (25%), 499 (25%).

### Example 5

The attachment of any acid to the 2′ or 3′ hydroxyl group on the A ring of the fluorescent dyes was accomplished using the methodology as described in Briggs et al. (1997) J. Chem. Soc., Perkin Trans., 1:1051.

### Example 6

Protocol for Nile Red Lipid Binding Assay

0,25 μl nile red dye stock solution (10 μM) was added to 90 μl HEPES buffer (final concentration 30 nM) and the mixture was transferred to a fluorimeter cuvette. For determining the submaximal concentration of dye binding to the protein, the latter was titrated until no further fluorescence increase at 620 nm could be detected.

For a typical lipid binding assay a protein concentration that showed 90% of the maximal fluorescence increase was selected. Increasing concentrations of lipid or drug were added to replace the dye in a dose-dependent fashion. Fluorescence values at 620 nm were corrected for basal fluorescence at 620 nm prior to protein addition as well as for dilution effects from adding increasing ligand concentrations. The starting fluorescence value minus the value at a given ligand concentration was plotted as % replacement.

1-21. (canceled)

22. A compound according to formula I 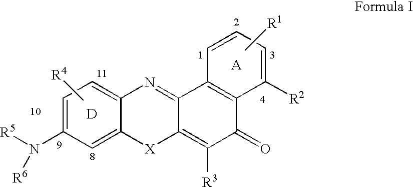
wherein
R¹ is independently in position 2′, 3′ or in both positions of the A ring and is hydroxyl, linear or branched, cyclic or acyclic —O-alkyl, wherein the alkyl group has 1-12 carbon atoms, —O-(alkyl)-COOH, wherein the alkyl group has 1- 12 carbon atoms, —O-(aryl)COOH, —O-aryl, linear or branched —O—(CH₂)ₘ—CH(NH₂)—COOH with m=1-6, or any combination thereof;
R⁴ is in position 8′, 10′, or 11′ of the D ring of the compound and is H or (CH₂)ₙ—CH₃ with n=0-6, COOH, CN, or halogen,
R⁵ and R⁶ independently are ethyl-, methyl-, H, or —(CH₂)₃ connected to position 8 or 10 of the D ring,
and wherein
provided that X is O,
R² is F, Cl, Br, I, H, CN, —CH═CH-alkyl, —C≡C-alkyl or aryl and
R³ is F, Cl, Br, I, CN, —CH=CH-alkyl, —C≡C-alkyl or aryl,
and
provided that X is S,
R² and R³ independently are F, Cl, Br, I, H, CN, —CH═CH-alkyl, —C≡C-alkyl or aryl.

23. The compound according to claim 22, wherein
R¹ is independently in position 2′, 3′ or in both positions of the A ring and is hydroxyl, linear or branched, cyclic or acyclic —O-alkyl, wherein the alkyl group has 3-8 carbon atoms, —O-(alkyl)-COOH, wherein the alkyl group has 3-8 carbon atoms, —O-(aryl)COOH, —O-aryl, linear or branched —O—(CH₂)ₘ—CH(NH₂)—COOH with m=2-4, or any combination thereof.

24. The compound according to claim 22,
wherein X is O, and
R⁴ is H.

25. The compound according to claim 22,
wherein X is S, and
R⁴ is methyl.

26. The compound according to claim 22 wherein R¹ is in position 2′ of the A ring and is —O—(CH₂)₃—CH₃, R² and R⁴ each are H, R³ is F, X is O, and R⁵ and R⁶ each are ethyl.

27. The compound according to claim 22, wherein
R² and R⁴ each are H,
R³ is Br,
X is O, and
R⁵ and R⁶ each are ethyl.

28. The compound according to claim 22, wherein
R² and R³ each are Br,
R⁴ is H,
X is O, and
R⁵ and R⁶ each are ethyl.

29. The compound according to claim 22, wherein
R² and R³ each are H,
R⁴ is methyl,
X is S, and
R⁵ and R⁶ each are ethyl.

30. The compound according to claim 22, wherein R⁵ and R⁶ each are H.

31. The compound according to claim 30, covalently bound via a spacer coupled to the amino group of the D-ring to a glass or plastic surface.

32. The compound according to claim 31, wherein the glass or plastic surface is the surface of a multi-well-plate, of a plate reader plate, of a micro array chip or of a microfluidic device.

33. A method of identifying lipid binding sites on proteins, which method comprises adding the protein to be tested to an aqueous solution of a fluorescent compound according to formula I 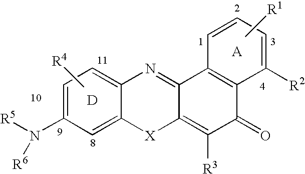
Formula I
wherein
R¹ is independently in position 2′, 3′ or in both positions of the A ring and is hydroxyl, linear or branched, cyclic or acyclic —O-alkyl, wherein the alkyl group has 1-12 carbon atoms, —O-(alkyl)-COOH, wherein the alkyl group has 1-12 carbon atoms, —O-(aryl)COOH, —O-aryl, linear or branched —O—(CH₂)ₘ—CH(NH₂)—COOH with m=1-6, or any combination thereof;
R² and R³ independently are F, Cl, Br, I, H, CN, —CH═CH-alkyl, —C≡C-alkyl, or aryl,
X is O or S,
R⁴ is in position 8′, 10′, or 11′ of the D ring of the compound and is H or (CH₂)ₙ—CH₃ with n=0-6, COOH, CN, or halogen
R⁵ and R⁶ independently are ethyl-, methyl-, H, or —(CH₂)₃ connected to position 8 or 10 of the D ring.

34. The method according to claim 33, wherein the binding of ligands to lipid binding sites of proteins is measured.

35. Assay for identifying lipid binding sites on proteins which comprises using the compound of formula I as defined in claim 22 comprising the steps of
dissolving a compound according to formula I in an aqueous solvent
adding the protein to be tested for containing a lipid binding site,
measuring the intensity of fluorescence emission at the wavelength of maximal emission of the fluorescent dye in non-aqueous solution.

36. The assay according to claim 35, wherein the compound of formula I is covalently linked to glass or plastic surfaces of multi well plates, plate reader plates, micro array chips or microfluidic devices, is applied, and wherein the intensity of fluorescence emission at the wavelength of maximal emission of the fluorescent dye is measured in an appropriate reader.

37. Competitive binding assay to measure the binding of ligands to lipid binding sites of proteins using the compound of formula I as defined in claim 22 comprising the steps of
dissolving the compound according to formula I in an aqueous solvent,
adding a protein containing at least one lipid binding site,
adding the ligand to be tested, and
measuring the intensity of fluorescence emission at the wavelength of maximal emission of the fluorescent dye in non-aquous solution.

38. The assay according to claim 37, wherein the compound of formula I is covalently linked to glass or plastic surfaces of multi well plates, plate reader plates, micro array chips or microfluidic devices, is applied, and wherein the intensity of fluorescence emission at the wavelength of maximal emission of the fluorescent dye is measured in an appropriate reader.

39. The method of studying pharmacokinetics of drug binding to the lipid binding sites of proteins, which method comprises the steps of the competitive binding assay described in claim 37.

40. The method of high throughput screening for ligands which are useful as competitive inhibitors of protein-lipid interactions, which method comprises the steps of the competitive binding assay described in claim 37.

41. Competitive inhibitors of protein lipid interactions identified by carrying out the assay according to claim 37.

42. Kit for identifying competitive inhibitors of a protein-lipid interaction comprising
the fluorescent compound of the formula I according to claim 22,
optionally a protein with at least one lipid binding site and
and optionally a solvent to solubilize the fluorescent dye and the protein.

43. A method of providing a medicament comprising a competitive inhibitor of a protein lipid interaction comprising the steps of
dissolving the compound according to formula I as defined claim 22 in an aqueous solvent,
adding a protein containing at least one lipid binding site,
adding the compound to be tested,
measuring the intensity of fluorescence emission at the wavelength of maximal emission of the fluorescent dye in non-aqueous solution,
identifying a compound which is a competitive inhibitor of the protein lipid interaction by detecting an alteration of the emission wavelength or fluorescence intensity of the compound according to formula I,
providing the same compound by chemical or biological methods
and manufacturing the compound into a medicament.

44. The Assay as according to claim 35, where the step of
adding the protein to be tested for containing a lipid binding site is in various concentrations.
